(19) United States
(12) Patent Application Publication
(10) Pub. No.: US 2022/0064378 A1
(43) Pub. Date: Mar. 3, 2022

(54) Star Polypeptides

(71) Applicant: The Royal College of Surgeons Ireland, 2 Dublin (IE)

(72) Inventors: Sally-Ann Cryan, 2 Dublin (IE); Andreas Heise, 2 Dublin (IE); David Walsh, 2 Dublin (IE); Joanne O'Dwyer, 2 Dublin (IE); Fergal O'Brien, 2 Dublin (IE); Garry Duffy, 2 Dublin (IE)

(21) Appl. No.: 17/415,454

(22) PCT Filed: Dec. 23, 2019
(86) PCT No.: PCT/EP2019/086910

§ 371 Date: Jun. 17, 2021

(30) Foreign Application Priority Data
Dec. 21, 2018 (GB) 1821014.6

(51) Int. Cl. C08G 73/02; A61K 45/06; A61K 38/17

(52) U.S. Cl. CPC C08G 73/028; A61K 38/1709; A61K 45/06

(57) ABSTRACT

A star polypeptide for use as a medicament, the star polypeptide comprising or consisting of a core and polypeptide arms radiating from the core. The star polypeptide may be used to deliver a therapeutic cargo and/or for its intrinsic properties. Therapeutic cargoes include a protein(e.g. VEGF); a nucleic acid (e.g. in vitro transcribed mRNA or microRNA); and/or a drug (e.g. diclofenac, azithromycin and/or rifampicin). Intrinsic properties include osteogenesis, angiogenesis and inhibition of bacteria.

### FIELD OF THE INVENTION

The present invention relates to new applications of star polypeptides, particularly new medical uses of star polypeptides, and also relates to compositions comprising star polypeptides.

### BACKGROUND OF THE INVENTION

Star polymers are a broad class of polymer architectures which consist of linear arms radiating from a central core. A “star shaped polypeptide” or “star polypeptide” or “star poly(amino acid) is a specific type of star-polymer whereby the “arms” consist of polypeptides. Star-shaped polypeptides may be formed via an N-carboxyanhydride (NCA) polymerisation reaction. NCAs are anhydrides of α-amino acids which can be synthesised, for example, via the use of chlorinating agents to cyclize the amino/carboxylic acid functionality on the α-amino carbon.

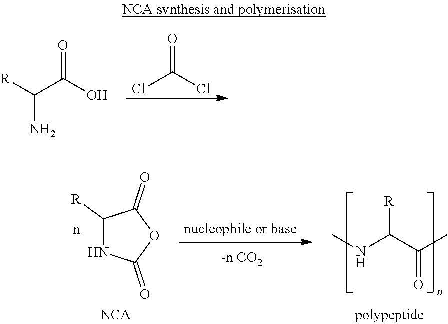

Following addition of a nucleophile the NCA ring structure undergoes a ring opening polymerisation (ROP) reaction which allows for the synthesis of polypeptides. This polymerisation reaction allows for tight control of the molecular weight of the resulting star-shaped polypeptides, forming unimolecular structures with a low polydispersity index.

Star-shaped polypeptides which have been synthesised to date include those having a core made from a polypropylene imine (PPI) dendrimer and arms formed using poly-L-lysine (Star-PLL), poly-L-glutamatic acid (Star-PGA), poly-L-arginine (Star-PLA) and poly-L-histidine (Star-PLH).

Byrne et al. (Biomater. Sci., 2013, 1, 1223) describes a series of star-shaped poly(lysine): G2(8)-PLL₄₀; G3(16)-PLL₄₀; G4(32)-PLL₄₀; G5(64)-PLL₄₀; G5(64)-PLL₅. Byrne et al. (2013) describes the transfection pDNA into Calu-3 cells using a star-shaped G5(64)-PLL₄₀ “polyplex”.

Byrne et al. (Macromolecular Rapid Communications, Vol. 36, 2015, pp. 1862-1876) is a review article describing the synthesis of certain star shaped polypeptides, and the opportunities for the delivery of therapeutics.

Walsh et al. (Mol. Pharmaceutics 2018, 15, 1878) describes the generation of three star-PLLs and also explains the nomenclature commonly used in the field: G3(16)PLL₄₀ (16-star-PLL) (G3=generation 3 polypropyleneimine dendrimer (PPI) core, (16)=16 polymeric L-lysine arms, 40=40 L-lysine subunits per arm, mol. weight=134 000 g/mol); G4(32)PLL₄₀ (32-star-PLL) (G4=generation 4 PPI core, (32)=32 polymeric L-lysine arms, 40=40 L-lysine subunits per arm, mol. weight=318 000 g/mol); and G5(64)PLL₅ (64-star-PLL) (G5=generation 5 PPI core, (64)=64 polymeric L-lysine arms, 5=5 L-lysine subunits per arm, mol. weight=94 000 g/mol). Walsh et al describes the delivery of pDNA to mesenchymal stem cells (MSC).

WO03064452 describes a method for the preparation of highly branched (hyperbranched) polyLys structures. This process does not allow for precise structural control. For example, it is not possible to control the number of branching points and the molecular weight between branching points. As such, WO'452 is not a good starting point for optimising therapeutic loading and transfection properties.

WO2018081845A1 discloses a star shaped peptide polymer comprising a multifunctional centre with a plurality of terminal arms extending therefrom. The terminal arms are statistical or random peptide copolymers of at least a cationic amino acid residue and a hydrophobic amino acid residue. The star shaped peptide is said to be useful in the treatment of a bacterial infection of the spleen.

### SUMMARY OF THE INVENTION

According to a first aspect of the invention there is provided a star polypeptide for use as a medicament,

the star polypeptide comprising or consisting of a core and polypeptide arms radiating from the core.

The star polypeptide may be for use to stimulate a response in a subject's body, such as to stimulate cell growth and/or tissue regeneration. Additionally or alternatively, the star polypeptide may be for use to inhibit the growth of bacteria. For example, a cationic star polypeptide may be for use to inhibit the growth of bacteria.

It will be appreciated that certain star-polypeptides have been described for use as gene delivery vectors (Byrne et al. 2013). However the inventors have determined that star polypeptides possess an intrinsic bioactivity profile when unbound to any therapeutic cargo. The examples demonstrate the osteogenic potential, angiogenic potential, and anti-bacterial potential of the star polypeptides themselves. Without being bound by theory, it is understood that the star polypeptide promotes cell growth and tissue generation due the ability to deliver a high density amino acid payload to cells, which is significantly more effective than delivery of disperse amino acids or linear polypeptides that are not provided by a star polypeptide in accordance with the invention. The anti-bacterial properties of the start polypeptides are understood to be in regard to the star polypeptide's ability to interfere and disrupt bacterial membranes, such as Escherischia coli and mycobacterial membranes, further such as the membrane of Mycobacterium tuberculosis.

Therefore, in one embodiment, the star polypeptide is used in the absence of another therapeutically active agent. The star polypeptide may not be arranged to carry or deliver a therapeutically active agent. The star polypeptide may not be bound to another therapeutically active agent. The star polypeptide may consist essentially of a core and polypeptide arms radiating from the core. The star polypeptide may not be arranged to deliver another molecule to, or into, cells or tissue.

In one embodiment, the therapeutically active agent may comprise any molecule, such as any biologically active molecule, that is not covalently bound to the star polypeptide. The therapeutically active agent may comprise a protein or peptide that is not covalently bound to the star polypeptide. The therapeutically active agent may comprise a non-small molecule. In another embodiment, the therapeutically active agent may comprise a small molecule, peptide, protein or nucleic acid. In another embodiment, the therapeutically active agent may comprise nucleic acid, such as siRNA, modified messenger RNAs (mRNAs), micro RNAs or DNA constructs. The therapeutically active agent may comprise a physiologically or metabolically relevant protein or nucleic acid.

### Cell and Tissue Growth

The star polypeptide may be for use to induce tissue regeneration, for example in the case of a tissue defect, damage or disease. The tissue may be bone tissue. The tissue may be cartilage tissue. The tissue may be selected from any one of the group comprising bone tissue, cartilage, skin tissue, such as dermis or epidermis; mucosal tissue; neuronal tissue; spinal tissue; organ tissue, such as pancreas tissue, or cardiac tissue; and ischeamic tissue; or combinations thereof.

In one embodiment, the star polypeptide may be for use to induce osteogenesis, i.e. to form bone. The star polypeptide may be for use in bone repair. As such, the star polypeptide may be for use to treat and/or prevent a range of conditions which affect bone formation including brittle bone disease (osteogenesis imperfecta), osteopenia and osteoporosis. The star polypeptide may be for use to facilitate the healing of bone fractures or to treat osteomyelitis. In another embodiment, the star polypeptide may be for use to facilitate the integration of osteogenic materials in the body. In another embodiment, the star polypeptide may be for use to facilitate dental implants, or to facilitate implants or tissue repair in craniofacial surgery, such as cleft lip and palate surgery.

In another embodiment, the star polypeptide may be for use to induce angiogenesis, i.e. to form blood vessels. As such, the star polypeptide may be employed to treat and/or prevent a range of conditions including cardiovascular diseases. In another embodiment, the star polypeptide may be for use to promote angiogenesis for tissue regeneration or replacement. In another embodiment, the star polypeptide may be for use to promote angiogenesis for wound repair, peripheral vascular disease, ischaemic disease or orthopaedic regeneration.

In an embodiment wherein the star polypeptide is used to promote cell or tissue growth (e.g. in angiogenesis, osteogenesis, tissue repair and the like), the star polypeptide may comprise about 64 polypeptide arms. In another embodiment wherein the star polypeptide is used to promote cell or tissue growth (e.g. in angiogenesis, osteogenesis, tissue repair and the like), the star polypeptide may comprise between about 40 and about 80 polypeptide arms. In another embodiment wherein the star polypeptide is used to promote cell or tissue growth (e.g. in angiogenesis, osteogenesis, tissue repair and the like), the star polypeptide may comprise between about 50 and about 72 polypeptide arms. In another embodiment wherein the star polypeptide is used to promote cell or tissue growth (e.g. in angiogenesis, osteogenesis, tissue repair and the like), the star polypeptide may comprise between about 60 and about 68 polypeptide arms. In another embodiment wherein the star polypeptide is used to promote cell or tissue growth (e.g. in angiogenesis, osteogenesis, tissue repair and the like), the star polypeptide may comprise between about 8 and about 80 polypeptide arms. In another embodiment wherein the star polypeptide is used to promote cell or tissue growth (e.g. in angiogenesis, osteogenesis, tissue repair and the like), the star polypeptide may comprise between about 8 and about 64 polypeptide arms. In another embodiment wherein the star polypeptide is used to promote cell or tissue growth (e.g. in angiogenesis, osteogenesis, tissue repair and the like), the star polypeptide may comprise between about 8 and about 40 polypeptide arms. In another embodiment wherein the star polypeptide is used to promote cell or tissue growth (e.g. in angiogenesis, osteogenesis, tissue repair and the like), the star polypeptide may comprise between about 8 and about 32 polypeptide arms. In another embodiment wherein the star polypeptide is used to promote cell or tissue growth (e.g. in angiogenesis, osteogenesis, tissue repair and the like), the star polypeptide may comprise between about 8 and about 20 polypeptide arms.

In an embodiment wherein the star polypeptide is used to promote cell or tissue growth (e.g. in angiogenesis, osteogenesis, tissue repair and the like), the star polypeptide may comprise an average polypeptide arm length of about 5 to about 20 amino acid residues. In another embodiment wherein the star polypeptide is used to promote cell or tissue growth (e.g. in angiogenesis, osteogenesis, tissue repair and the like), the star polypeptide may comprise an average polypeptide arm length of between about 20 and about 100 amino acid residues. In another embodiment wherein the star polypeptide is used to promote cell or tissue growth (e.g. in angiogenesis, osteogenesis, tissue repair and the like), the star polypeptide may comprise an average polypeptide arm length of between about 5 and about 100 amino acid residues.

### Use as an Antibacterial

The star polypeptide may be employed for its bacteriostatic effect, i.e. to inhibit bacterial growth. As such, the star polypeptide may be for use to treat and/or prevent a bacterial infection.

The star polypeptide may be employed to inhibit gram positive bacteria, such as Staphylococcus. The Staphylococcus may comprise Staphylococcus newman (a methicillin sensitive Staphylococcus) or a methicillin resistant Staphylococcus aureus.

In another embodiment, the star polypeptide may be for use to treat or prevent a gram negative bacterial infection, such as E. coli. The E. coli may comprise CTF-resistant E. coli strains.

In another embodiment, the bacterial infection may be a Staphylococus infection, such as S. aureus or S. epidermidis. In one embodiment, the bacterial infection may be a Mycobacterium infection, such as M. tuberculosis.

In an embodiment wherein the star polypeptide is used as an antibacterial agent, the star polypeptide may comprise about 32 polypeptide arms. In another embodiment wherein the star polypeptide is used as an antibacterial agent, the star polypeptide may comprise between about 20 and about 40 polypeptide arms. In another embodiment wherein the star polypeptide is used as an antibacterial agent, the star polypeptide may comprise between about 28 and about 36 polypeptide arms. In another embodiment wherein the star polypeptide is used as an antibacterial agent, the star polypeptide may comprise between about 30 and about 34 polypeptide arms.

In an embodiment wherein the star polypeptide is used as an antibacterial agent, the star polypeptide may comprise an average polypeptide arm length of about 5 to about 20 amino acid residues. In another embodiment wherein the star polypeptide is used as an antibacterial agent, the star polypeptide may comprise an average polypeptide arm length of between about 20 and about 100 amino acid residues. In another embodiment wherein the star polypeptide is used as an antibacterial agent, the star polypeptide may comprise an average polypeptide arm length of between about 5 and about 100 amino acid residues.

The star polypeptide may be for use as an antimicrobial to treat a material, for example a surgical implant prior to implantation.

Therefore, according to another aspect of the present invention, there is provided the use of a star polypeptide as an antibacterial agent ex-vivo.

The use may be as a disinfectant. The star polypeptide may be applied to a surface, such as the surface of an implant, instrument or medical device.

According to another aspect of the present invention there is a method of disinfecting a material, the method comprising applying a star polypeptide to the material.

The material to be treated or disinfected may be an instrument or medical device. In another embodiment, the material may be a surgical device. In one embodiment, the material to be treated or disinfected with the star polypeptide is an implant.

The term “disinfecting” in the context of a material is understood to mean a reduction or a complete killing of viable microbes, such as bacteria on the material.

The present invention has advantageously been found to be intrinsically anti-bacterial and can help to treat or prevent infections in vivo, and help to disinfect medical equipment, surfaces and implants, where there is an increasing need to combat resistant strains of bacteria.

### Therapeutic Cargo Delivery

In addition to the intrinsic properties of star polypeptides to promote cell or tissue growth, and as an antibacterial, the star polypeptides are also capable of carrying and delivering another molecule or multiple molecules (i.e. a cargo). Therefore, the star polypeptide may be a delivery vehicle/carrier.

Therefore, according to another aspect of the invention there is provided a star polypeptide for use as a medicament, the star polypeptide comprising a core and polypeptide arms radiating from the core, wherein the star polypeptide is arranged to deliver a cargo to a cell or tissue of a subject.

According to another aspect of the present invention, there is provided a method of modifying cellular function in a subject or a cell culture comprising administering a composition comprising star polypeptides, optionally with a cargo, in accordance with the invention.

The cell culture may be in vitro.

The cargo may be a therapeutic cargo. For example a cargo that is arranged to treat or prevent a disease or condition. In one embodiment, the cargo is a small molecule or a biological molecule. The cargo may comprise a protein; a nucleic acid; or a drug (e.g. a small molecule of less than 900 Da); or combinations thereof. In one embodiment, the cargo is a protein or peptide. In another embodiment, the cargo is a nucleic acid.

The cargo, such as a protein, may be cationic or anionic. In an embodiment wherein the cargo, such as a protein, is cationic, the polypeptide arms of the star polypeptide may be anionic. Alternatively, if the cargo, such as a protein, is anionic, the polypeptide arms of the star polypeptide may be cationic. In an embodiment wherein the cargo is nucleic acid, such as DNA, the star polypeptide may comprise cationic polypeptide arms, thereby facilitating the complexing of the nucleic acid with the star polypeptide(s). In another embodiment wherein the cargo is nucleic acid, such as DNA, the star polypeptide may comprise a majority of cationic polypeptide arms relative to anionic and/or neutral polypeptide arms, thereby facilitating the complexing of the nucleic acid with the star polypeptide(s).

In one embodiment, the cargo may not be covalently bound to the star polypeptide. The cargo may comprise a protein or peptide that is not covalently bound to the star polypeptide. The cargo may be electrostatically bound to the star polypeptide(s).

The cargo may comprise a physiologically or metabolically relevant protein or nucleic acid. The cargo may comprise an intracellular protein. The cargo may comprise a signal protein, which is a protein involved in a signal pathway. The cargo may comprise a protein involved with regulation of expression or metabolism of a cell. The cargo may comprise a protein involved with cell division. The cargo may comprise a protein involved with cell differentiation, such as stem cell differentiation. The cargo may comprise a protein required for induction of pluripotent stem cells. The cargo may comprise a protein involved with cardiac cell differentiation. The cargo may comprise a marker, such as a protein marker. The cargo may comprise a bacterial, or bacterially derived protein. The cargo may comprise a mammalian, or mammalian derived protein. The cargo may be any peptide, polypeptide or protein. The cargo may comprise research, diagnostic or therapeutic molecules. The cargo may comprise a transcription modulator, a member of signal production. The cargo may comprise an enzyme or substrate thereof, a protease, an enzyme activity modulator, a perturbimer and peptide aptamer, an antibody, a modulator of protein-protein interaction, a growth factor, or a differentiation factor. The cargo may be a protein arranged to be post-translationally modified within the cell. The cargo may be arranged to be functional once inside the cell. For example, the cargo may not be functional until after delivery into the cell.

The cargo may comprise any intracellular molecule. The cargo may comprise any protein or molecule having an intracellular function (mode of action), intracellular receptor, intracellular ligand, or intracellular substrate. The cargo may comprise a protein or molecule that is naturally/normally internalised into a cell. The cargo may comprise a protein intended for delivery or display in the cell surface, such as a cell surface receptor. The cargo may be selected from any of the group comprising a therapeutic molecule; a drug; a pro-drug; a functional protein or peptide, such as an enzyme or a transcription factor; a microbial protein or peptide; and a toxin; or nucleic acid encoding thereof.

In one embodiment, the cargo may comprise a transcription factor, or a nucleic acid encoding a transcription factor. Additionally or alternatively, the cargo may comprise a growth factor or a nucleic acid encoding a growth factor.

The growth factor may comprise a growth factor selected from the group comprising adrenomedullin (AM); angiopoietin (Ang); autocrine motility factor; bone morphogenetic protein (BMP); ciliary neurotrophic factor (CNTF); Leukemia inhibitory factor (LIF); interleukin-6 (IL-6); colony-stimulating factor; macrophage colony-stimulating factor (M-CSF); granulocyte colony-stimulating factor (G-CSF); granulocyte macrophage colony-stimulating factor (GM-CSF); epidermal growth factor (EGF); ephrin; erythropoietin (EPO); fibroblast growth factor (FGF); glial cell line-derived neurotrophic factor (GDNF); neurturin; persephin; artemin; growth differentiation factor-9 (GDF9); hepatocyte growth factor (HGF); hepatoma-derived growth factor (HDGF); insulin; insulin-like growth factor; interleukin; keratinocyte growth factor (KGF); migration-stimulating factor (MSF); macrophage-stimulating protein (MSP), also known as hepatocyte growth factor-like protein (HGFLP); myostatin (GDF-8); neuregulin; neurotrophin; brain-derived neurotrophic factor (BDNF); nerve growth factor (NGF); neurotrophin; placental growth factor (PGF); platelet-derived growth factor (PDGF); renalase (RNLS); anti-apoptotic survival factor; T-cell growth factor (TCGF); thrombopoietin (TPO); transforming growth factor; transforming growth factor alpha (TGF-α); transforming growth factor beta (TGF-β); tumor necrosis factor-alpha (TNF-α); vascular endothelial growth factor (VEGF); and Wnt, or combinations thereof, and/or nucleic acid encoding such growth factors.

The cargo may comprise nucleic acid that upregulates, or may be capable of upregulating, a growth factor in the cell. In one embodiment, the cargo may comprise nucleic acid that upregulates, or may be capable of upregulating, BMP2 and/or VEGF expression in the cells.

In one embodiment, the cargo comprises Vascular Endothelial Growth Factor (VEGF, an anionic protein) or a nucleic acid encoding VEGF.

In an embodiment wherein the cargo comprises nucleic acid, the nucleic acid may comprise DNA, such as plasmid DNA (pDNA), small interfering RNA (siRNA) and/or micro-RNA (miRNA). The nucleic acid may comprise viral nucleic acid. The nucleic acid may comprise one or more gene encoding sequences, and/or non-coding regulatory sequences. The nucleic acid may encode a protein described herein.

In one embodiment, the cargo may comprise a nucleic acid-editing molecule, such as a

CRISPR-Cas molecule. The CRISPR-Cas molecule may comprise CRISPR-Cas9. The nucleic acid editing molecule may comprise a complex of a nucleic acid editing enzyme and a guide nucleic acid. The guide nucleic acid may be targeted to a sequence or gene of interest associated with a disease or condition described herein.

In one embodiment, the cargo may comprise DNA encoding a gene. The gene may be for use in ex vivo cellular engineering. In one embodiment, the gene may encode chimeric antigen receptor (CAR), for example to be inserted into T-cells in CAR-T therapy.

When the star polypeptide comprises G3(16)PLL₄₀ (16-star-PLL), G4(32)PLL₄₀ (32-star-PLL), or G5(64)PLL₅ (64-star-PLL) and the therapeutic cargo comprises a nucleic acid, the nucleic acid may comprise miRNA. In one embodiment, the cargo does not comprise pDNA and/or siRNA.

The drug may be a hydrophilic drug or a hydrophobic drug. The drug may be doxorubicin (CAS 23214-92-8). The drug may be a small molecule, such as an anti-cancer, anti-inflammatory or anti-infective agent.

The cargo may have a molecular weight of at least 1 kDa. The cargo may have a molecular weight of at least 5 kDa. The cargo may have a molecular weight of at least 10 kDa. The cargo may have a molecular weight of at least 20 kDa. The cargo may have a molecular weight of 400 KDa or less. The cargo may have a molecular weight of 300 kDa or less. The cargo may have a molecular weight of between about 0.5 kDa and about 400 kDa. In another embodiment, the cargo may have a molecular weight of between about 0.1 kDa and about 400 kDa. In another embodiment, the cargo may have a molecular weight of between about 100 Da and about 900 Da.

Where the cargo comprises amino acids, the cargo may be between about 20 and about 30,000 amino acids in length. The cargo may be between about 20 and about 10,000 amino acids in length. The cargo may be between about 20 and about 5,000 amino acids in length. The cargo may be between about 20 and about 1000 amino acids in length. The cargo may be at least about 20 amino acids in length. The cargo may be at least about 100 amino acids in length.

The cell may be a mammalian cell, such as a human cell. The cell may be a cancerous cell. The cell may be a stem cell. The cell may be a mutant cell. The cell may comprise a population of cells. The population of cells may be a mixed population of cell types. In one embodiment the cell or population of cells are isolated from a subject and/or other cell types. The cells may be part of a tissue or whole organ, which may be in-situ, or ex-situ from the body. The cells may be in a cell culture in vitro. The cell may be a stem cell, such as a mesenchymal stem cell. The cell may be part of a cell line, such as Calu-3 airway cells, cystic fibrosis bronchial epithelial cells, and A549 adenocarcinoma airway epithelial cells.

The star polypeptides and the cargo may be provided in a ratio of 1:1 star polypeptide:cargo. In another embodiment, the star polypeptides may be provided in a ratio of between 0.01:1 and 500:1 relative to the cargo. In another embodiment, the star polypeptides may be provided in a ratio of between 1:1 and 100:1 relative to the cargo. In another embodiment, the star polypeptides may be provided in a ratio of between 5:1 and 50:1 relative to the cargo. In another embodiment, the star polypeptides may be provided in a ratio of about 5:1 relative to the cargo. In another embodiment, the star polypeptides may be provided in a ratio of about 50:1 relative to the cargo. For example in an embodiment wherein the cargo is nucleic acid, the ratio of star polypeptides to nucleic acid may be at least 1:1, or between 1:1 and 50:1.

The invention also resides in compositions comprising the star polypeptide and the therapeutic cargo. The invention also resides in compositions comprising the star polypeptide without a therapeutic cargo.

The composition may be a pharmaceutically acceptable composition. The composition may comprise pharmaceutically acceptable excipients. The composition may comprise the star polypeptides, with or without cargo, in a carrier. The carrier may be saline, or a buffer. The carrier may be a hydrogel or a scaffold, such as a porous scaffold.

The star polypeptides, with or without cargo, may be provided in a therapeutically effective amount. The therapeutically effective amount may comprise a dose of at least about 30 μg/kg. In another embodiment, the therapeutically effective amount may comprise a dose of at least about 10, 15, 20, 25 or 30 μg/kg. In another embodiment, the therapeutically effective amount may comprise a dose of between about 10 μg/kg and about 100 μg/kg.

In an embodiment comprising the use of the star polypeptide, such as the 64-star-PLL, for bone regeneration, the dose may be at least 30 μg/kg.

In an embodiment comprising the use of the star polypeptide, such as the 32-star-PLL, as an antibacterial, the star polypeptide may be provided at a concentration of at least 2 μg/ml. In another embodiment comprising the use of the star polypeptide, such as the 32-star-PLL, as an antibacterial, the star polypeptide may be provided at a concentration of at least 7 μg/ml. In another embodiment comprising the use of the star polypeptide, such as the 32-star-PLL, as an antibacterial, the star polypeptide may be provided at a concentration of at least 20 μg/ml. In another embodiment comprising the use of the star polypeptide, such as the 32-star-PLL, as an antibacterial, the star polypeptide may be provided at a concentration of at least 3 μM, or at least 3.13 μM.

In an embodiment comprising the use of the star polypeptide, such as the 32-star-PLL, as an antibacterial against Staphylococcus (such as S. newman), the star polypeptide may be provided at a concentration of at least 2 μg/ml. In another embodiment comprising the use of the star polypeptide, such as the 32-star-PLL, as an antibacterial against MRSA, the star polypeptide may be provided at a concentration of at least 7 μg/ml. In another embodiment comprising the use of the star polypeptide, such as the 32-star-PLL, as an antibacterial against E. coli, the star polypeptide may be provided at a concentration of at least 20 μg/ml. In another embodiment comprising the use of the star polypeptide, such as the 32-star-PLL, as an antibacterial against M. tuberculosis, the star polypeptide may be provided at a concentration of at least 3 μM, or at least 3.13 μM.

In an embodiment wherein the star polypeptides, or compositions thereof, are administered, they may be administered topically, systemically, orally, intravenously, or subcutaneously. The star polypeptides may be injected or seeded onto a tissue site for treatment. In another embodiment, the star polypeptides, or compositions thereof, may be inhaled, for example using a nebuliser.

### Nebuliser

The star polypeptide for use as a medicament may be delivered by means of an aerosol, for example using a nebuliser. As such, the star polypeptide may be for use to treat a subject's respiratory system. For example the use may be treatment for a respiratory disease. The respiratory disease may be asthma, cystic fibrosis, chronic obstructive pulmonary disease (COPD), alpha-1-antitrypsin deficiency, idiopathic pulmonary fibrosis or lung cancer.

According to another aspect of the invention there is provided a composition comprising a star polypeptide and (i) a protein, (ii) a nucleic acid and/or (iii) a drug, the star polypeptide comprising a core and polypeptide arms radiating from the core.

According to another aspect of the invention there is provided a composition comprising a star polypeptide and (i) a scaffold; and/or (ii) a hydrogel; the star polypeptide comprising a core and polypeptide arms radiating from the core.

In one embodiment, the scaffold is a collagen scaffold. In another embodiment, the scaffold may be a polymer scaffold, such as a synthetic polymer scaffold. The scaffold may comprise one or more natural polymers such as collagen, hyaluronic acid, gelatin, alginate, silk, or chitosan. The scaffold may be a porous, fibrous or tubular scaffold, or a particulate or film-based structure from the micro to the macro scale. The scaffold may be freeze-dried, electrospun or woven, self-assembled or solvent casted or 3D printed. The scaffold may comprise bioactive glass and/or ceramic. In one embodiment, the scaffold is biodegradable. In another embodiment, the scaffold is non-degradable, for example made from poly(methyl)acrylates, polyurethanes, or polyacrylamides.

The hydrogel may be a hyaluronic acid hydrogel for example. In one embodiment, the hydrogel may be a clay nanoparticle hydrogel, such as a natural or synthetic layered silicate hydrogel. For example, the clay nanoparticle may be a synthetic hectorite (also known as Laponite). The clay nanoparticle gel may further comprise a polymer, such as hyaluronic acid polymer. The hydrogel may be polyacrylate-based or polysaccharide-based. In one embodiment, the hydrogel is biodegradeable. In one embodiment, the hydrogel may comprise or consist of natural polymers selected from chitosan, alginate, cellulose, gelatin, fibrin, hyaluronic acid, dextran, and collagen, or combinations thereof. Additionally or alternatively, the hydrogel may comprise synthetic material selected from poly(ethylene glycol), and methylacrylamide, hydroxyethylmethacrylate, or combinations thereof. The skilled person will recognise a number of hydrogel and scaffold forming materials may be used.

In one embodiment, the hydrogel may be considered a scaffold and vice versa. In one embodiment, the hydrogel may be considered to be a drug depot.

The invention also resides in a method for the preparation of the composition of this aspect.

According to another aspect of the present invention, there is provided a method of tissue repair or replacement in a subject, the method comprising the administration of a star polypeptide to the subject.

According to another aspect of the present invention, there is provided a method of controlled-drug release in a subject, the method comprising the administration of a star polypeptide to the subject, wherein the star polypeptide carries, or is complexed with, a cargo as described herein.

According to another aspect of the present invention, there is provided a method of treatment or prevention of a bacterial infection in a subject, the method comprising the administration of a star polypeptide to the subject.

According to another aspect of the present invention, there is provided a method of gene therapy or genetic engineering comprising the delivery of a star polypeptide and cargo, or a composition comprising such a star polypeptide and cargo, to a cell, wherein the cargo comprises nucleic acid.

In one embodiment, the cell is in vitro. In another embodiment, the cell is in vivo.

The nucleic acid may:
encode a gene and/or a regulatory sequence; or
be a guide nucleic acid for a nucleic acid-editing protein, and the cargo further comprises the nucleic acid-editing protein.

In one embodiment, the gene encodes chimeric antigen receptor (CAR). The cell may be a T-cell.

According to another aspect of the invention there is provided a composition comprising a star polypeptide, the star polypeptide comprising a core and polypeptide arms radiating from the core,
wherein the polypeptide arms comprise (i) poly-L-lysine and (ii) poly-L-arginine and/or poly-L-histidine.

According to another aspect of the present invention, there is provided a kit, the kit comprising:
a star polypeptide described herein;
a cargo described herein and/or one of a nebuliser, hydrogel or tissue scaffold.

### DETAILED DESCRIPTION OF THE INVENTION

The star polypeptide for use in the invention can be described with reference to the core and the polypeptide arms. The core can be considered to be a multifunctional core molecule. The core may be a dendrimer core, such as a polypropylene imine (PPI) or polyethylenimine (PEI) dendrimer core. Other potential dendrimer cores include poly(amidoamine) (PAMAM) dendrimer, trimethylol propane (bis-MPA) dendrimer and dendritic polylysine.

A dendrimer is a repetitively branched molecule. Dendrimers often adopt a spherical three-dimensional morphology. Dendrimers can be classified by generation, which refers to the number of repeated branching cycles that are performed during synthesis. For example, if a dendrimer is made by convergent synthesis, and the branching reactions are performed onto the initial molecule three times, the resulting dendrimer is considered a third generation dendrimer. Each successive generation results in a dendrimer having a molecular weight which is approximately twice that of the previous generation.

Alternatively, the core might be a hyperbranched core such as trimethylol propane (bis-MPA) (Boltron™) or polyester amide (Hybrane®) or polyether amines (Jeffamine®) or hyper branched polylysine.

Alternatively, the core might be a linear core such as polylysine, polyacrylate, polymethacrylate, polyester, polyamide with pending functional groups.

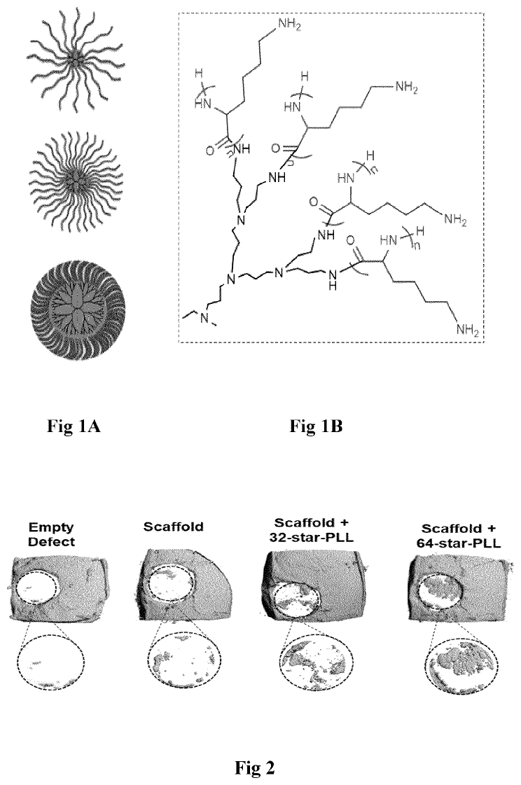

FIG. 1A illustrates three star shaped polypeptides formed using a PPI dendrimer core and poly-L-lysine (PLL): G3(16)PLL₄₀, G4(32)PLL₄₀ and G5(64)PLL₅. Each structure contains a different core generation size (generation 3, generation 4 or generation 5), number of poly-L-lysine arms (16 arms, 32 arms or 64 arms) and number of poly-L-lysine subunits per arm (5 subunits or 40 subunits). FIG. 1B is the branched, repeating arm structure showing four poly-L-lysine arms, each containing “n” repeating L-lysine subunits.

PPI dendrimers are also known as DAB-Am-x dendrimers, where DAB represents the diaminobutane “hub” and x=4, 8, or 16 etc. for the number of primary amine end groups associated with the generations 1, 2, or 3, respectively. The PPI dendrimer core may be first generation (1G, C₁₆H₄₀N₆), second generation (G2, C₄₀H₉₆N₁₄), third generation (G3, C₈₈H₂₈₈N₃₀), fourth generation (G4), fifth generation (G5), sixth generation (G6) or seventh generation (G7). The present invention exemplifies star polypeptides having second, third, fourth, fifth and sixth generation PPI dendrimer cores.

A polypeptide is a chain of amino acid monomers linked by peptide (amide) bonds.

The star polypeptide comprises polypeptide arms radiating from the core. The star polypeptide may comprise 8 or more, 16 or more, 32 or more or 64 or more peptide arms and/or the star polypeptide may comprise 128 arms or fewer, 64 arms or fewer, 32 arms or fewer, 16 arms or fewer or 8 arms or fewer. The star polypeptide may comprise from 8 to 64 arms.

A polypeptide arm can be described with reference to its length (the number of amino acid subunits per arm) and/or its composition (the type or types of amino acid present). A polypeptide arm can consist of a single type of amino acid or may comprise mixtures of amino acids. For example, an arm may comprise up to 5 different types of amino acids. The amino acids may be arranged in a random fashion or in a block sequence.

The star polypeptide may comprise 1 or more, 2 or more, 3 or more, 5 or more, 10 or more, 15 or more, 20 or more, 25 or more, 30 or more, 35 or more, 40 or more amino acid subunits per arm and/or the star polypeptide may comprise 50 or fewer, 40 or fewer, 30 or fewer, 20 or fewer, 15 or fewer, 10 or fewer or 5 or fewer amino acid subunits per arm. The number of subunits per arms can be determined from the ratio of the components prior to polymerisation, as is common in polymer chemistry.

The polypeptide arms may comprise or consist of natural amino acids. In another embodiment, the polypeptide arms may comprise or consist of non-natural amino acids. The arms may comprise poly-L-alanine, poly-L-arginine, poly-L-aspargine, poly-L-aspartic acid, poly-L-cysteine, poly-L-glutamine, poly-L-glutamic acid, poly-L-glycine, poly-sarcosine, poly-L-histidine, poly-L-isoleucine, poly-L-leucine, poly-L-lysine, poly-L-methionine, poly-L-phenylalanine, poly-L-proline, poly-L-serine, poly-L-threonine, poly-L-tryptophan, poly-L-tyrosine and/or poly-L-valine.

In one embodiment the arms comprise poly-L-arginine, poly-L-glutamic acid, poly-L-histidine and/or poly-L-lysine.

In one embodiment the arms comprise poly-L-arginine, poly-L-glutamic acid, poly-L-histidine, poly-sarcosine and/or poly-L-lysine.

The star polypeptide may be a star-shaped homopolymer, where all of the arms consist of one type amino acid. More complex arrangements, such as star-shaped random and block co-polymers can also be generated thereby allowing tailoring of the star-shaped polypeptide for the specific cargo.

In one embodiment the star polypeptide is a star-shaped homopolymer having arms consisting of poly-L-lysine. The star polypeptide may comprise G3(16)-PLL₂₀-co-PLA₂₀; G4(32)-PLL₂₀-co-PLH₂₀; G4(32)-PLL₂₀-co-PLA₂₀; G5(64)-PLL₅-co-PLH₅; and/or G5(64)-PLL₅-co-PLA₅. The inventors believe these random co-polymers to be new.

The star polypeptide may comprise a star shaped block co-polymer, wherein the polypeptide arms comprise an inner portion (closer to the core) and an outer portion (further from the core). The inner portion may comprise or consist of poly-L-lysine, poly-L-alanine or poly-L-threonine for example. The outer portion may comprise or consist of poly-L-valine, poly-L tryptophan or poly-L-glutamic acid for example.

Exemplary star polypeptides having a PPI dendrimer core are set out in the table below.

| | | Core | Number | Number of amino |
|---|---|---|---|---|
| Structure | Amino Acid | Generation | of arms | acid subunits/arm* |
| G3(16)-PLL₂₀-co-PLA₂₀ | L-Lysine/L-Arginine | 3rd | 16 | 20 |
| | *co = random copolymer | | | |
| G4(32)-PLL₂₀-co-PLH₂₀ | L-Lysine/L-Histidine | 4th | 32 | 20 |
| | *co = random copolymer | | | |
| G4(32)-PLL₂₀-co-PLA₂₀ | L-Lysine/L-Arginine | 4th | 32 | 20 |
| | *co = random copolymer | | | |
| G5(64)-PLL₅-co-PLH₅ | L-Lysine/L-Histidine | 5th | 64 | 5 |
| | *co = random copolymer | | | |
| G5(64)-PLL₅-co-PLA₅ | L-Lysine/L-Histidine | 5th | 64 | 5 |
| | *co = random copolymer | | | |
| G2(8)-PLL₄₀ | L-Lysine | 2nd | 8 | 40 |
| G3(16)-PLL₄₀ | L-Lysine | 3rd | 16 | 40 |
| G4(32)-PLL₄₀ | L-Lysine | 4th | 32 | 40 |
| G5(64)-PLL₄₀ | L-Lysine | 5th | 64 | 40 |
| G5(64)-PLL₅ | L-Lysine | 5th | 64 | 5 |
| G2(8)-PLG₆₀ | L-Glutamic acid | 2nd | 8 | 60 |
| G2(8)-PLG₄₀ | L-Glutamic acid | 2nd | 8 | 40 |
| G2(8)-PLG_{12.5} | L-Glutamic acid | 2nd | 8 | 13 |
| G3(16)-PLG₄₀ | L-Glutamic acid | 3rd | 16 | 40 |
| G3(16)-PLG_{6.3} | L-Glutamic acid | 3rd | 16 | 6 |
| G4(32)-PLG₄₀ | L-Glutamic acid | 4th | 32 | 40 |
| G4(32)-PLG_{3.1} | L-Glutamic acid | 4th | 32 | 3 |
| G5(64)-PLG₄₀ | L-Glutamic acid | 5th | 64 | 40 |
| G5(64)-PLG₂₀ | L-Glutamic acid | 5th | 64 | 20 |
| G5(64)-PLG_{7.5} | L-Glutamic acid | 5th | 64 | 8 |
| G5(64)-PLG_{1.6} | L-Glutamic acid | 5th | 64 | 2 |

*as predicted from the starting materials

As explained above, the star polypeptide has intrinsic bioactivity. In addition, the star polypeptide can be used to deliver a therapeutic cargo. The inventors propose that the polypeptide arms can be tailored to suit the specific application.

In particular, the polypeptide arms will have a charge which can be varied by changing the amino acid subunits. The use of positive (e.g. cationic) polypeptide arms is considered useful for entering cells, and thereby providing an antimicrobial effect. This is demonstrated in the examples by the use of star polypeptides comprising poly-L-lysine arms to inhibit bacterial growth.

The use of polypeptide arms having a charge (whether positive or negative) is considered beneficial as compared to neutral polypeptide arms.

pKₐ is the log₁₀ of an acidity constant Kₐ of an acid HA, such as an amino acid. The acid HA dissociates into A⁻, the conjugate base, and H⁺, a hydrogen ion. pKₐ is determined as pKₐ=[HA]/[A³¹ H⁺] where square brackets represent the concentration of the species at equilibrium. pKₐ depends on temperature and pressure, which is SATP (25° C. and 100 kPa) unless stated otherwise. In one embodiment, the star polypeptide has a pKa of 7 or less. In one embodiment, the star polypeptide has a pKa of about 6.5. In another embodiment, the star polypeptide may have a pKa of between 5 and 7, or between 5.5 and 7, or between 6 and 7. The skilled person will recognise that the amino acid content of the polypeptide arms contributes to the pKa of the star polypeptide, and they can be adjusted accordingly to provide a desired pKa profile. For example in an embodiment wherein the star polypeptide is intended for intracellular entry, the star polypeptide may be designed to have a pKa of less than 7.

Arginine, histidine and lysine have positively charged side chains, whereas aspartic acid and glutamic acid have negatively charged side chains.

The length of the polypeptide arms can be varied. It is proposed that longer arms provide greater mobility.

The star polypeptides comprising high density, short cationic arms, for example 64-star-PLL (i.e. having 64 arms of 5 AA length) may be optimal for intracellular cargo delivery, such as delivery of nucleic acid. Polyanionic arms can provide encapsulation of positively charged therapeutic cargoes, such as VEGF. For antibacterial applications a cationic component may be provided. Addition of a hydrophobic component may enhance such attributes. In addition, longer side chains may provide for better antimicrobial activity, for example 32-star-PLL (having 40AA long side chains).

Advantageously, 64-star-PLL with short 5AA long arms has a lower pKa, for example of 6.5, than those observed for longer polylysine arms. This provides that this structure has greater potential to act as a proton sponge in an acidic lysosome compared to structures with longer polyamino acid side chains and thereby improves its efficiency as a cargo delivery vector. In addition using live cell imaging it was observed that the 64-star PLL taken up by mammalian cells was rapidly processed and juxtanuclear localisation of pDNA was evident. Such effect may be due to the high lysine concentration of the star-PLLs functioning as a nuclear localisation signal within the cell.

The invention is further described, in a non-limiting manner, with reference to the following examples in which:

FIG. 1 is a graphical overview of a range of star-shaped polypeptides formed using poly-L-lysine;

FIG. 2 shows an assessment of the osteogenic potential of the star-PLL vector: representative microCT scans of a rodent calvarial defect, four weeks post implantation of either (A) no treatment -empty defect, (B) a collagen-hydroxyapatite scaffold only, (C) a collagen-hydroxyapatite scaffold loaded with the 32-star-PLL structure and (D) a collagen-hydroxyapatite scaffold loaded with the 64-star-PLL structure;

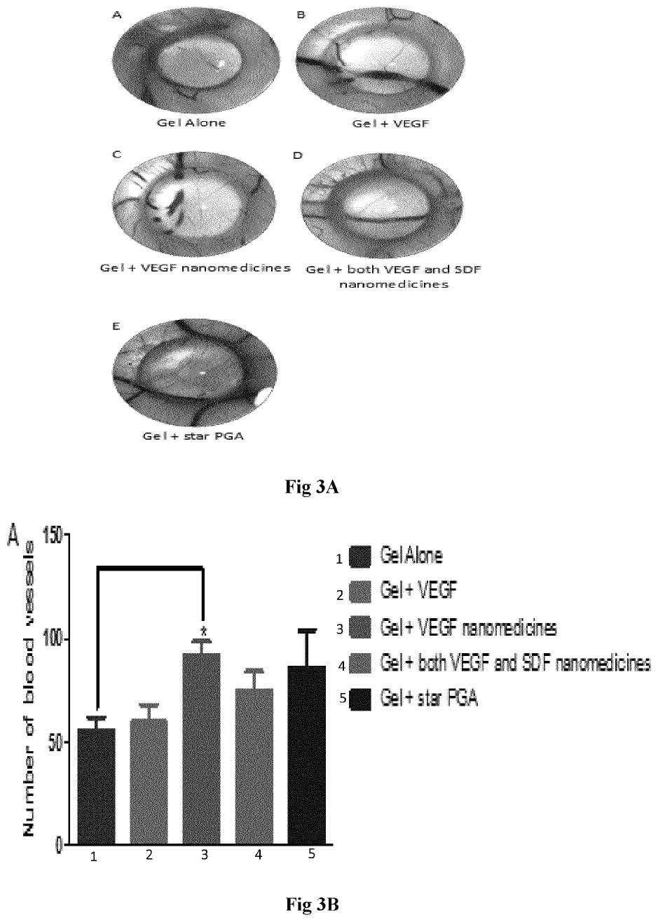

FIG. 3A shows a summary of a chick chorioallantoic membrane study where the following groups were assessed: (A) hyaluronic acid hydrogel (“hydrogel”), (B) the hydrogel loaded with VEGF protein, (C) the hydrogel loaded with star-PGA-VEGF complexes, (D) the hydrogel loaded with a dual combination of star-PGA-VEGF and star-PGA-SDF complexes and (E) the hydrogel loaded with star-PGA (no therapeutic cargo). The number of blood vessels formed are quantified in FIG. 3B (SDF=stromal derived factor);

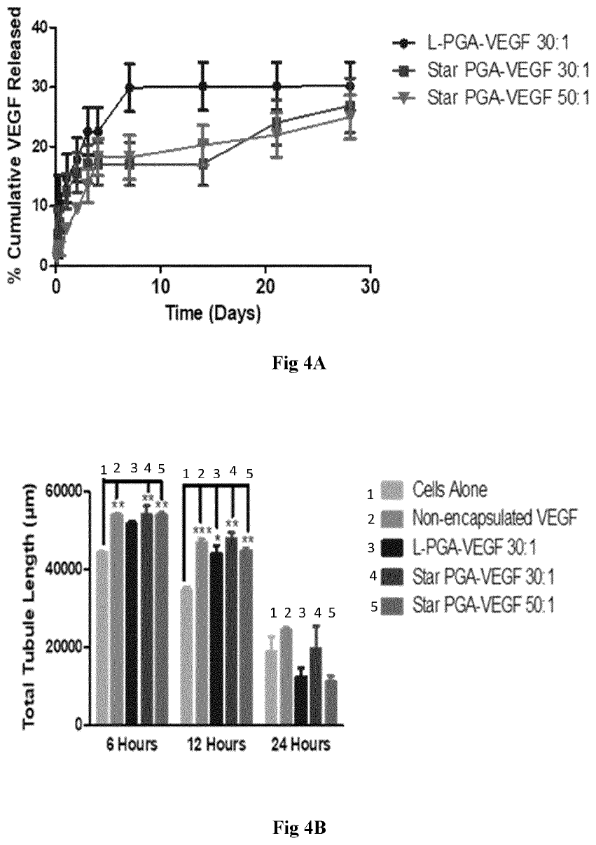

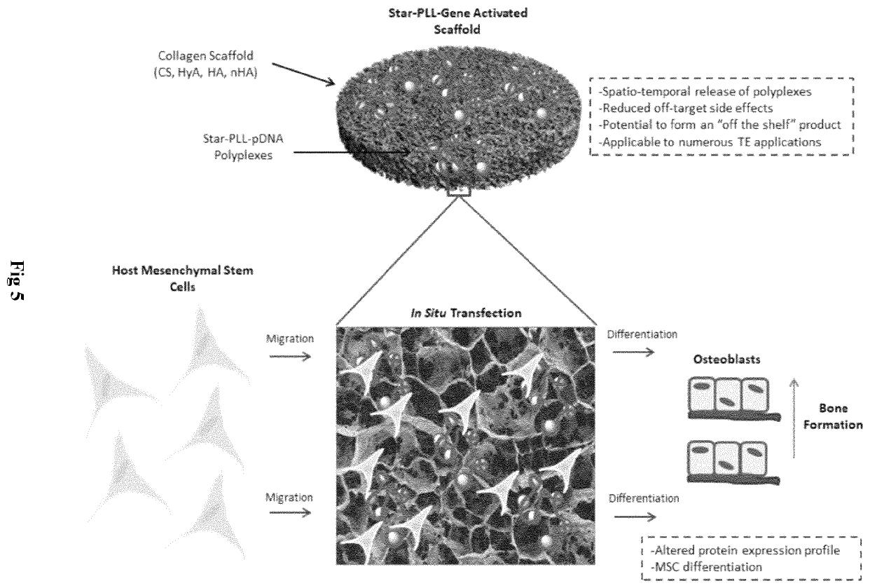

FIG. 4A shows VEGF released from linear poly-L-glutamic acid bound VEGF (L-PGA-VEGF) or star PGA-VEGF formulations over 28 days. The star PGA-VEGF formulations exhibited sustained VEGF release while the L-PGA-VEGF formulation did not; FIG. 4B shows the bioactivity of VEGF released from PGA-VEGF nanoparticles—Quantification of tubule lengths, confirming significantly increased tubule lengths with all VEGF containing groups at 12 hours. n=3; *p<0.05, **p<0.01, ***p<0.001;

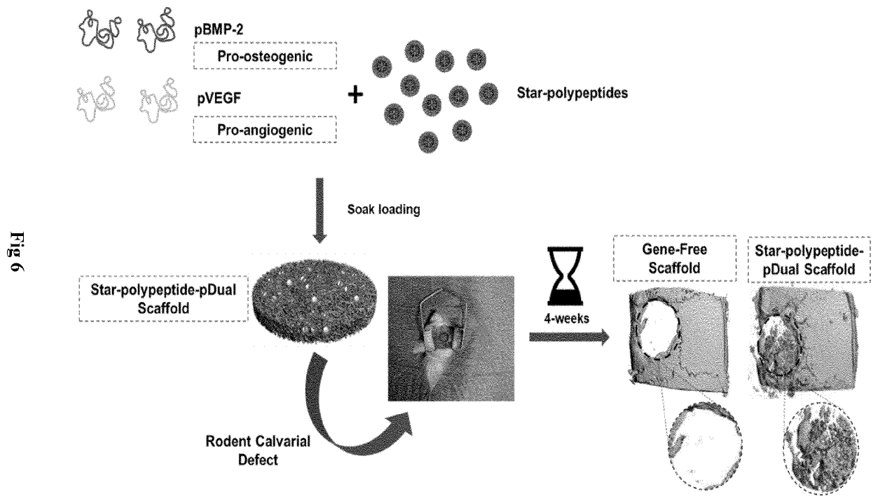

FIG. 5 is a schematic diagram of the star-PLL-pDNA gene activated scaffold; and

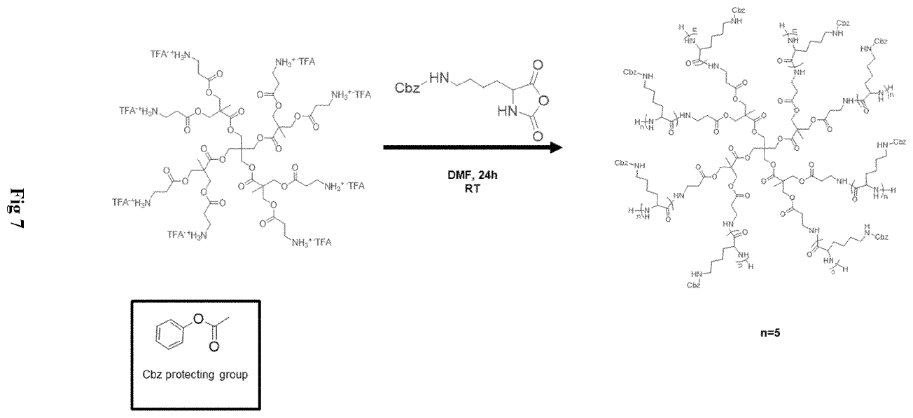

FIG. 6 is an overview of the pre-clinical work completed using bone tissue repair as a primary application. A collagen-hydroxyapatite scaffold was soak loaded with the 64-star-PLL containing both pVEGF and pBMP-2 and the resultant gene activated scaffold was implanted into a critical sized, rodent calvarial defect. Four weeks post implantation, the defect was assessed, and enhanced levels of new bone tissue had formed using the gene activated scaffold compared to the gene free scaffold.

FIG. 7 shows the synthesis of G1(8)-BisMPANH₄⁺TFA⁻-PZLL₄₀.

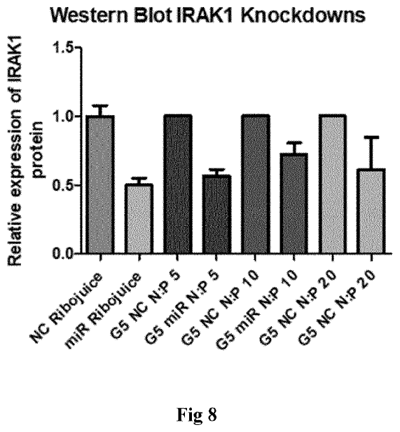

FIG. 8 demonstrates the ability of G5(64)-PLL₅ to deliver microRNA (miR-146a), to cystic fibrosis bronchial epithelial cells (CFBEs).

### Intrinsic Bioactivity

The inventors have determined that the star-shaped polypeptide structure possesses an intrinsic bioactivity profile when unbound to any therapeutic cargo. Without being bound by theory, the inventors hypothesize that this bioactive profile is due to the presentation of a high density of amino acids on the star-shaped polypeptide at the cell surface.

The bioactive nature is demonstrated for osteogenic potential, angiogenic potential, and anti-bacterial potential.

### Osteogenic Potential

The amino acid used for synthesis is poly-L-lysine and the exemplary star-shaped polypeptide is a generation 5 polypropyleneimine (PPI) dendrimer with 64-poly-L-lysine arms and 5 poly-L-lysine subunits per arm (64-star-PLL=G5(64)-PLL₅ FIG. 1A). Here, using both in vitro and in vivo experiments we have demonstrated that the star-PLL vector is capable of inducing osteogenesis when unbound to a therapeutic nucleic acid cargo. In vivo, when both the 32-star-PLL (G4(32)-PLL40) and 64-star-PLL vectors were loaded onto a collagen-hydroxyapatite scaffold and implanted into a cranial defect in a rodent, they resulted in enhanced new bone volume within the defect at 4-weeks compared to a collagen-hydroxyapatite scaffold alone or an empty defect without a scaffold (FIG. 2). These findings highlight the osteogenic potential of the star-PLL vector within a complex in vivo environment. It should be noted however, that the osteogenic potential of the system can be further augmented by complexing the star-PLL with a therapeutic plasmid cargo (See Integration into 3D scaffolds below).

### Angiogenic Potential

The amino acid used for synthesis is poly-L-glutamic acid and the exemplary star-shaped polypeptide is a generation 2 PPI dendrimer with 8-poly-L-glutamic acid arms and 40 poly-L-glutamic subunits per arm (Star-PGA=G2(8)-PLL₄₀).

Following the incorporation of star-PGA into a hyaluronic acid hydrogel, the inventors determined that the star-PGA structure increases the number of blood vessels formed compared to a hydrogel alone in an in vivo chick chorioallantoic membrane model when unbound to a therapeutic protein cargo (FIG. 3).

Importantly, when the total length of blood vessels formed in this model were quantified as a marker of angiogenesis, the gel containing only the star-PGA molecules resulted in a comparable angiogenic effect to that of a gel containing star-PGA-VEGF complexes. This finding strongly suggests that the star-PGA structure possess an angiogenic potential.

### Antibacterial Potential

The amino acid in use is poly-L-lysine and the specific structure investigated was the 32-star-PLL (G4(32)-PLL₄₀, FIG. 1). Antimicrobial peptides (AMPs) are peptides which are capable of interacting with microbial membranes through electrostatic interactions and physically damage the bacterial morphology. These AMPs have been widely regarded as a promising solution to combat the emergence of multi-drug resistant (MDR) bacteria, as unlike traditional antibiotics, bacteria are less likely to develop resistance to AMPs. To date, few promising AMP candidates exist due to their high toxicity in mammalian cells.

In this preliminary work, the inventors determined that the 32-star-PLL structure is inhibitory towards bacterial growth (i.e. the star-PLL structure is bacteriostatic). Thus far, the inventors demonstrated this bacteriostatic effect in both gram positive (Staphylococcus new man (a methicillin sensitive Staphylococcus) and a methicillin resistant Staphylococcus aureus) and gram negative (CTF E. coli) strains (Table below).

| | Minimum 32-star-PLL concentration to | |
|---|---|---|
| Strain | inhibit bacterial growth | |
| Staph. newman | 2 μg/ml | (6.28 nM solution) |
| MRSA | 7 μg/ml | (22 nM solution) |
| E. coli | 20 μg/ml | (62 nM solution) |

| | |
|---|---|
| Mycobacterium tuberculosis | 3.13 μM |

In addition to common bacterial infections, tuberculosis remains a major public health concern, with ˜9.5 million new cases per year and a need for new preventative and treatment approaches. The inventors also determined that the 32-star-PLL (G4(32)-PLL₄₀), but not the 64-star-PLL (G5(64)-PLL₅) can inhibit the growth of Mycobacterium Tuberculosis.

Without being bound by theory, the inventors propose that there is a fine balance to be struck between the size, charge and mobility of the polypeptide arms. For example, 32 star-PLL may have greater mobility than 64 star-PLL due to the presence of longer arms—40 amino acid subunits per arm compared to 5 amino acids per arm.

Importantly, the concentrations determined here for the inhibition of bacterial growth using the star-PLL structure are below the threshold concentration which causes toxicity in mammalian cells thereby suggesting the star-PLL structure can overcome one of the principal current limitations for classic AMPs.

### Delivery of Therapeutic Cargoes

The inventors have demonstrated that the star-polypeptides can be used to deliver both nucleic acids and proteins to multiple cell types, each of which is discussed individually below. Without being bound by theory, the inventors propose that the star-shaped polypeptides could also be used for the delivery of small molecules e.g. Doxorubicin.

### Nucleic Acid Delivery

The star-shaped polypeptide functions as a non-viral gene delivery vector for delivery of nucleic acids to cells and poly-L-lysine is the exemplary amino acid used to form its “arms”.

This yields a “star-shaped poly-L-lysine polypeptide” or “star-PLL”. Within the star-PLL subfamily, three structures have been extensively explored. These are:
1: G3(16)PLL₄₀ (16-star-PLL)
2: G4(32)PLL₄₀ (32-star-PLL)
3: GS(64)PLL₅ (64-star-PLL)

Poly-L-lysine in a linear form was one of the first non-viral vectors studied for the delivery of nucleic acids to cells. It is a linear cationic polypeptide of the basic amino acid lysine which can interact with and electrostatically condense nucleic acids. While linear poly-L-lysine (L-PLL) is capable of forming nano-sized complexes with nucleic acids and protecting these nucleic acids from degradation by serum nucleases, complexes formed are polydisperse, often forming large aggregates thereby resulting in erratic cellular transfection. Furthermore, L-PLL suffers from a low transfection efficiency, a fact believed to be attributable to its poor buffering capacity thus preventing endosomal escape within the cell.

In contrast, the structure of the star-shaped poly-L-lysine polypeptides, with their densely packed poly-L-lysine units confers several advantages as a functional non-viral gene delivery vector compared to commonly used vectors such as L-PLL or commercially available systems. These advantages include:

Inexpensive, short (˜3 days), “clean” fabrication process which can be easily scaled.

Structural versatility which allows design/tailoring of the specific star-polypeptide structure, its molecular weight and number/length of attached polypeptide arms to the specific nucleic acid cargo being delivered. Theoretical ability to generate a library of these structures to suit the end user's requirements.

Ease of handling for the end user as supplied as a lyophilised powder which can be stored at room temperature and easily transported.

Ability to rapidly (<5 minutes) self-assemble with multiple nucleic acid cargos (plasmid DNA, siRNA, miRNA) to form nano-sized complexes in a simple “one tube” reaction.

Ability to efficiently deliver different nucleic acid cargos (plasmid DNA (pDNA), siRNA, miRNA) to both primary cells (e.g. mesenchymal stem cells) and cell lines (e.g. Calu-3 cells, cystic fibrosis bronchial epithelial cells, A549 s).

Transfection efficiency is superior or comparable to current gold standard/commercially available vectors such as polyethylenimine or Superfect™.

Ability to co-deliver two plasmids within a single population of star-PLLs thereby reducing the overall total dose of each plasmid required for therapeutic efficacy. This is facilitated by the high loading capacity of the star-polypeptide structure.

Ability to lyophilise star-PLL-nucleic acid complexes to form an “off the shelf”, ready to use transfection product.

When bound to a nucleic acid cargo, star-PLLs can protect this cargo from biological degradation in vitro against DNase, serum and heparan sulphate.

Optimised star-PLL-nucleic acid formulations possess favourable toxicity profiles in their respective cell types in vitro.

The inventors propose that the star-PLL structure can condense any cationic nucleic acid or combination of nucleic acids. The choice of specific star-shaped polypeptide structure will dictate the subsequent loading capacity of the formulation i.e. higher arm number structures and greater arm length structures can condense larger quantities of nucleic acid.

The star-PLL group of vectors have demonstrated efficacy for the delivery of multiple nucleic acid types to both primary cells and cell lines as shown in the table below:

| Nucleic Acid Types Delivered | Cell Types |
|---|---|
| Plasmid DNA (pDNA) | Mesenchymal Stem Cells |
| | THP-1 Human Monocytic Cells |
| Small Interfering RNA | Calu-3 Airway Epithelial Cells |
| (siRNA) | A549s Adenocarcinoma Airway |
| | Epithelial Cells |
| Micro-RNA (miRNA) | Cystic Fibrosis Bronchial Epithelial |
| | Cells |

Notably, the inventors have determined using an extensive and systematic screening process, optimum formulation conditions for the delivery of each nucleic acid type using the star-PLL vectors. For example, optimal delivery of pDNA to Mesenchymal Stem Cells, a difficult to transfect cell type can be achieved using a 64-star-PLL vector complexed with a relatively low pDNA dose (1 μg pDNA) and an N/P ratio (ratio of nitrogen in star-PLL to phosphates in pDNA) of 5. Similar screening processes have elucidated optimal formulation parameters for the delivery of siRNA and miRNA to the various cell types outlined above.

FIG. 8 demonstrates the ability of G5(64)-PLL₅ to deliver microRNA, specifically miR-146a, to cystic fibrosis bronchial epithelial cells (CFBEs) and elicit the desired effect on the protein expression of IRAK in the cells.

The inventors have demonstrated the use of a star polymer for the preparation of in vitro transcribed messenger RNA (IVT-mRNA) nanomedicines.

In vitro transcribed messenger RNA (IVT-mRNA) has become a promising alternative to other forms of nucleic acids in gene therapy. Unlike the commonly used plasmid DNA, mRNA does not require nuclear entry to be transcribed, leading to faster and higher protein expression levels. Star polymers (G5(64)-PLL₅) were able to successfully form cationic polyplexes from N/P ratios 2-20 with IVT-mRNA. The average diameter of the polyplexes was <200 nm across the various N/P ratios, demonstrating good potential for cell transfection.

### Protein Delivery

The star-shaped polypeptide functions as a carrier for the delivery of anionic proteins. In this example, the core material is a generation 2 polypropyleneimine dendrimer, the arms are formed using the polypeptide poly-L-glutamic acid and each arm contains approximately 40 repeating glutamic acid units. This yields a “star-shaped poly-L-glutamic acid polypeptide” or “star-PGA”.

The inventors developed the star-PGA structure specifically for the delivery of the anionic protein Vascular Endothelial Growth Factor (VEGF), but consider it plausible for the delivery of any anionic protein. VEGF is one of the most potent mediators of angiogenesis within the body and is envisaged as a powerful potential therapeutic for the recapitulation of the ischaemic myocardium post myocardial infarction. However, one of the main issues with the exploitation of VEGF for this application is its rapid degradation in vivo.

Electrostatic complexation of VEGF with the star-PGA structure at ratios of 30:1 & 50:1 (star-PGA:VEGF) results in the formation of nano-sized complexes with improved stability and a prolonged release profile. This star-shaped polypeptide retains all the handling/storage/scalable advantages discussed for the star-PLL structures above and confers several advantages for the specific delivery of VEGF (or other proteins) which include:

Effective encapsulation (>99.9%) of VEGF protein into the star-PGA structure.

Prolonged release of VEGF protein from the star-PGA structure over a 28-day period in vitro, with no release detected thereafter. FIG. 4A shows the VEGF released from linear poly-L-glutamic acid bound VEGF (L-PGA-VEGF) or star PGA-VEGF formulations over 28 days. The star PGA-VEGF formulations exhibited sustained VEGF release while the L-PGA-VEGF formulation did not.

VEGF which is released from the star-PGA structure retains its bioactivity and can induce the same degree of tubule formation (a marker of angiogenesis) in Human Umbilical Vein Endothelial Cells (HUVECs) compared to uncomplexed VEGF. FIG. 4B) shows bioactivity of VEGF released from PGA-VEGF nanoparticles—

Quantification of tubule lengths—confirming significantly increased tubule lengths with all VEGF containing groups at 12 hours. n=3; *p<0.05, **p<0.01, ***p<0.001.

Integration into 3D Scaffolds/Medical Devices for Controlled/Targeted Delivery & Tissue Regeneration The star-shaped polypeptides can be integrated into 3D scaffolds for tissue repair or into medical devices for site specific delivery. The inventors have successfully integrated star-shaped polypeptides into three principal scaffolds/devices: collagen based scaffolds (advanced in vivo studies complete); hyaluronic acid hydrogels (preliminary in vivo studies complete); and nebuliser devices (early developmental stage).
Collagen Based Scaffolds The star-shaped polypeptide in use are G4 (32-star) PLL and G5 (64-star) PLL. The collagen-based scaffolds were developed by SurgaColl Technologies (Dublin, Ireland). Extensive data was gathered on the use of the star-PLL structure to deliver pDNA from a collagen-based scaffolds, thereby forming a functional “gene activated scaffold” for the repair of various tissues. The premise of a gene activated scaffold for tissue repair is outlined in the schematic shown in FIG. 5.

Functional gene activated scaffolds can be formed via the incorporation of star-PLL-pDNA complexes (also referred to as polyplexes) into one of five different collagen scaffolds (collagen alone, collagen-chondroitin sulfate (CS), collagen-hyaluronic acid (HyA), collagen-hydroxyapatite (HA) and collagen-nanohydroxyapaite (nHA)).

Following implantation into the tissue defect, host mesenchymal stem cells will migrate throughout the scaffold structure and become transfected with the pDNA cargo, thereby causing them to begin the tissue repair process. In the schematic, a gene activated scaffold for bone tissue repair is illustrated. The star-PLL-pDNA complexes contain a pDNA cargo which will cause MSCs to differentiate down an osteogenic lineage, thereby resulting in the rapid formation of new bone tissue.

The star-PLL structure has been used to form a gene activated scaffold for bone tissue repair, with pre-clinical studies in a rodent model complete. The main findings are summarised below:

Star-PLL-pDNA complexes can be successfully incorporated into five different collagen based scaffolds (Collagen, Collagen-Chondroitin Sulfate (CS), Collagen-Hyaluronic Acid (HyA), Collagen-Hydroxyapatite (HA) and Collagen-nano hydroxyapatite (nHA)). Incorporation was confirmed using Scanning Electron Microscopy and Confocal Imaging.

When star-PLL-pDNA complexes are incorporated into collagen-scaffolds, the scaffold functions as a depot of pDNA complexes, prolonging their release over a 28-day period. In comparison, the use of uncomplexed pDNA in the scaffold (no star-PLL present) results in a burst release of pDNA from the scaffold by ˜72 hours.

Star-PLL-pDNA complexes which have been incorporated into collagen based scaffolds remain bioactive, capable of transfecting MSCs and altering gene expression both in vitro and in vivo.

Star-PLL-pDNA gene activated scaffolds are non-toxic both in vitro and in vivo.

Star-PLL-pDNA gene activated scaffolds can be lyophilised and stored to form an “off-the-shelf” product.

Using a rodent calvarial defect model, a 64-star-PLL-pDNA collagen-hydroxyapatite scaffold was capable of significantly increasing new bone formation at a 4-week timepoint compared to a gene free collagen-hydroxyapatite scaffold. In this instance, the star-PLL was dual loaded with two therapeutic plasmids of pVEGF and pBMP-2 (FIG. 6).

The figure illustrates an overview of the pre-clinical work completed using bone tissue repair as a primary application. Here, a collagen-hydroxyapatite scaffold was soak loaded with the 64-star-PLL containing both pVEGF and pBMP-2. The resultant gene activated scaffold was implanted into a critical sized, rodent calvarial defect. Four weeks post implantation, the defect was assessed, and enhanced levels of new bone tissue had formed using the gene activated scaffold compared to the gene free scaffold.

### Hyaluronic Acid Hydrogels

The star-shaped polypeptide in use is G2(8)-PLL₄₀ (the star-PGA), the therapeutic cargo is VEGF protein and the final application is to deliver VEGF to the ischemic myocardium post myocardial infarction. The inventors have gathered extensive data on the use of the star-PGA structure to deliver VEGF from hyaluronic acid based hydrogels (obtained from CONTIPRO®). The main findings are summarised below:

Star-PGA-VEGF complexes can be successfully incorporated into hyaluronic acid based hydrogels without compromising the hydrogel structure.

The distribution of star-PGA-VEGF complexes is homogenous throughout the hydrogel structure.

The star-PGA-VEGF loaded hyaluronic acid hydrogel can be successfully delivered using a syringe system whilst retaining integrity of the formulation.

The incorporation of star-PGA-VEGF complexes into the hyaluronic acid hydrogels allows for a prolonged release pattern, with ˜70% of complexes being released over a 35-day period.

Using an in vivo, chick chorioallantoic membrane assay, an enhanced number of blood vessels were observed using a hyaluronic acid gel loaded with the star-PGA-VEGF complexes compared to a non-loaded hyaluronic gel (FIG. 3a).

### Nebuliser Devices

The star-shaped polypeptide in use is the star-PLL polypeptide or the hybrid material formed using the amino acids poly-L-arginine (40% of arms) and poly-L-lysine (60% of arms) (PLL-PLA). This work was carried using a proprietary, high performance nebuliser device developed by a specialist respiratory company Aerogen (Galway, Ireland) but is not limited to this specific nebuliser device.

The respiratory system is one of the largest and highly specialised organs within the body and as a result it is an important emerging target for gene and protein based therapies. There is growing demand for therapies which can effectively and safely treat the range of chronic and acute diseases which affect the lungs such as asthma, cystic fibrosis, alpha-1-antitrypsin deficiency and lung cancer among many others. Due to the large surface area of the lungs, inhalation offers immense potential for both local and systematic delivery of a cargo, as discussed above, such as for gene, protein, or drug therapeutics.

The inventors have demonstrated that complexes formed with pDNA using the star-PLL or star-PLL-PLA structures can be successfully integrated into the nebuliser device and maintain their physicochemical characteristics (complex size and charge) following nebulisation. This finding suggests that the strong electrostatic attraction between the star-shaped polypeptide and its nucleic acid cargo permits its passage through an inhalation device thereby increasing the potential applications of star-polypeptides to include site specific respiratory delivery.

### Further Star Polypeptides for use in the Invention.

The inventors propose the use of star polypeptides having a bis-MPA core.

### Synthesis of G1(8)-BisMPAOH-PZLL₄₀

The NCA of c-carbobenzyloxy-L-lysine (ZLL) (0.6 g, 1.96 mmol) was added to a Schlenk tube. Under a nitrogen atmosphere 6 mL of anhydrous CHCl₃ was added, then 1mL DMF was added into suspension of monomer and CHCl₃. When the NCA was dissolved, a solution of G1-BisMPAOH dendrimer (29 mg, 4.89×10⁻⁵ mmol) in 2 mL DMF was quickly charged to solution via a syringe. The solution was allowed to stir for 24 h at room temperature. The polymer was precipitated into an excess of cold diethyl ether and dried under vacuum.

### Synthesis of G1(8)-BisMPANH₄⁺TFA⁻-PZLL₄₀

Referring to FIG. 7, the NCA of ε-carbobenzyloxy-L-lysine (ZLL) (1 g, 3.27 mmol) was added to a Schlenk tube. Under a nitrogen atmosphere 10 mL of anhydrous CHCl₃ was added, then 2 mL DMF was added into suspension of monomer and CHCl₃.

When the NCA was dissolved, a solution of G1-BisMPANH₄⁺TFA⁻ dendrimer (78 mg, 8.10×10⁻⁵ mmol) in 2 mL DMF was quickly charged to the solution via a syringe. The solution was allowed to stir for 24 h at room temperature. The polymer was precipitated into an excess of cold diethyl ether and dried under vacuum.

The inventors propose that the MPA core would replace the PPI core described above and could then be applied to both the biomaterials and the delivery applications that have already been exemplified. The polypeptide arms on the core can be tailored as already discussed.

The inventors propose the use of star polypeptides having a poly(sarcosine) hydrophilic shell. Polysarcosine offers the potential to effectively deliver therapeutic cargoes to mucosa e.g. gastrointestingal, respiratory, nasal mucosa where they will enhance mucus penetration, which the inventors have demonstrated with in vitro assays.

### Synthesis of Star Polymers PBLG-b-PSar (s-PBLG-b-PSar)

BLG=beta lactoglobulin

BLG NCA (0.04 g, 0.15 mmol) was dissolved in a mixture of DMF and CHCl₃ in a ratio 1:2 (2 mL) under a nitrogen atmosphere to a pre-dried Schlenk tube equipped with a magnetic stirrer. A solution of G2 PPI dendrimer (6 mg, 0.0077 mmol) in 1 mL CHCl₃ was quickly charged to the dissolved NCA solution via syringe. The solution was stirred at room temperature for 2 hours and it was periodically degassed under vacuum. FT-IR was used to monitor the complete consumption of the monomer. Then, a solution of the Sar NCA (0.56 g, 4.9 mmol) in DMF (3 mL) was prepared and charged to the reaction solution via syringe. The obtained polymer was 0.34 g. (yield: 55%).

Example of Star Polymer used to Encapsulate Small Molecules with a Range of Different Physicochemical Properties.

Star polypeptides, star-PLL and star-PGA, have been used to encapsulate a range of small molecule therapeutics with iterative design of the star polymer required in order to enable effective drug-loading. Therapeutic cargoes successfully encapsulated to-date include antibiotics and anti-inflammatories: diclofenac (G3(16)-PLL₂₀), azithromycin (G5(64)-PLL₅) and rifampicin (G3(16)-PLL₂₀).

1. A method of stimulating a response in a subject or in a cell culture comprising the step of:
administering a composition to the subject or to the cell culture, the composition comprising a star polypeptide and optionally a therapeutic cargo, the star polypeptide comprising or consisting of a core and polypeptide arms radiating from the core, thereby stimulating a response in the subject or in the cell culture.

2. The method according to claim 1, wherein the star polypeptide delivers a therapeutic cargo to the cell culture or to tissue of the subject, thereby stimulating the response in the subject or in the cell culture.

3. The method according to claim 2, wherein the therapeutic cargo comprises a protein; a nucleic acid; and/or a drug.

4. The method according to claim 2, wherein the therapeutic cargo comprises in vitro transcribed mRNA or microRNA.

5. The method according to claim 2, wherein the therapeutic cargo comprises Vascular Endothelial Growth Factor.

6. The method according to claim 2, wherein the therapeutic cargo comprises diclofenac, azithromycin and/or rifampicin.

7. The method according to claim 2, wherein the therapeutic cargo is delivered to a stem cell.

8. The method according to claim 1, wherein the composition is administered to the subject or to the cell culture in the absence of another therapeutically active agent.

9. (canceled)

10. The method according to claim 1, wherein administering the composition to the subject stimulates cell growth and/or induces tissue generation.

11. The method according to claim 10, wherein administering the composition to the subject induces osteogenesis or angiogenesis.

12. The method according to claim 1, wherein administering the composition to the subject inhibits the growth of bacteria.

13. (canceled)

14. The method according to claim 12, wherein the bacteria is selected from E. coli, Staphylococus and/or Mycobacterium.

15. The method according to claim 1, wherein the composition is administered to the subject by means of a nebuliser, a hydrogel or a scaffold.

16. A composition comprising a star polypeptide and (i) a protein, (ii) a nucleic acid and/or (iii) a drug, the star polypeptide comprising a core and polypeptide arms radiating from the core.

17. A composition comprising a star polypeptide and (i) a scaffold and/or (ii) a hydrogel; the star polypeptide comprising a core and polypeptide arms radiating from the core.

18. The method according to claim 1, wherein the polypeptide arms comprise (i) poly-L-lysine and (ii) poly-L-arginine and/or poly-L-histidine.

19. (canceled)

20. The method according to claim 1, wherein the core is a polypropylene imine dendrimer core.

21. The method according to claim 1, wherein the star polypeptide comprises (i) from 8 to 64 polypeptide arms and/or (ii) from 5 to 40 amino acid subunits per arm.

22. The method according to claim 1, wherein the arms comprise poly-L-arginine, poly-L-glutamine, poly-L-glutamic acid, poly-L-histidine, poly-sarcosine and/or poly-L-lysine.

23. The method according to claim 1, wherein the star polypeptide comprises G2(8)-PLL₄₀, G5(64)-PLL₅, G3(16)-PLL₂₀-co-PLA₂₀; G4(32)-PLL₂₀-co-PLH₂₀; G4(32)-PLL₂₀-co-PLA₂₀; G5(64)-PLL₅-co-PLH₅; and/or G5(64)-PLL₅-co-PLA_{5.}
